(19) United States
(12) Patent Application Publication
(10) Pub. No.: US 2013/0102650 A1
(43) Pub. Date: Apr. 25, 2013

(54) ANTIMICROBIAL COMPOUNDS OF 1,4-NAPHTOQUINONE STRUCTURE

(71) Applicants: Yap Jean-Bertrand Boum, II, Mbarara (UG); Tamara Basta-Le-Berre, Orsay Cedex (FR); Ursula Liebl, Palaiseau (FR); Hannu Myllykallio, Palaiseau (FR)

(72) Inventors: Yap Jean-Bertrand Boum, II, Mbarara (UG); Tamara Basta-Le-Berre, Orsay Cedex (FR); Ursula Liebl, Palaiseau (FR); Hannu Myllykallio, Palaiseau (FR)

(21) Appl. No.: 13/640,187

(22) PCT Filed: Apr. 8, 2011
(86) PCT No.: PCT/EP2011/055537

§ 371 Date: Jan. 2, 2013

(30) Foreign Application Priority Data
Apr. 9, 2010 (EP) 10305369.0

(51) Int. Cl. C07C 50/32; C07C 211/42; C07C 50/38; C07D 333/22

(52) U.S. Cl. CPC C07C 50/32; C07D 333/22; C07C 211/42; C07C 50/38
USPC 514/438; 514/681; 514/657; 552/298; 549/72; 552/297

(57) ABSTRACT

The present invention relates to a compound having formula (I) wherein: R₁ is chosen from the group consisting of: phenyl group, possibly substituted, —CH₂—C₆H₄—R′₁ group, R′₁ being chosen from the group consisting of: H, —OH, halogen, alkyl, aryl, CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group, R′₁ being preferably in para position, and —CH₂—CO—R′ group, R′ representing an aryl or heteroaryl group, said aryl and heteroaryl groups being possibly substituted, R₂ is chosen from the group consisting of: —OH and halogen, and R₃, R₄, R₅ and R₆ are in particular H, for its use for the prevention and/or the treatment of bacterial infections.

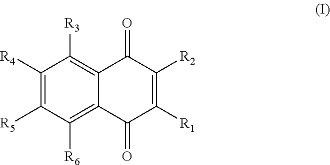

The present invention concerns new antimicrobial compounds having a 1,4-naphtoquinone structure.

The growing problem of antibiotic resistant bacteria (see Chambers H. F., Emerg. Infect. Dis. 7:178-182 (2001); Hecht D. W., Clin. Infect. Dis. 39:92-97 (2004); Jacobs M. R., Am. J. Med. 1 17 Suppl. 3A:3S-15S (2004); Molbak K., Clin. Infect. Dis. 41:1613-1620 (2005); Shah et al., Res. Microbiol. 155:409-421 (2004); Wisplinghoff et al., Clin. Infect. Dis. 39:309-317 (2004); and Zinner S. H., Expert Rev. Anti. Infect. Ther. 3:907-913 (2005)) points to a need for new anti-infective therapies. However, the rate of new antimicrobial is unlikely to meet the expected need for the foreseeable future.

Deoxythymidine 5′-monophosphate (thymidylate or dTMP), an essential DNA precursor, can be synthesized in the cell either from simpler molecules (bicarbonate, aspartate and glutamine) or, in some species, dTMP precursors can be salvaged from the growth medium. The last step of de novo thymidylate synthesis is the methylation of deoxyuridine 5′-monophosphate (uridylate or dUMP) to dTMP (J. S. Finer-Moore, D. V. Santi, R. M. Stroud, Biochemistry 42, 248 (Jan. 21, 2003). This methylenetetrahydrofolate (CH₂H₄folate)-dependent methylation reaction is catalyzed by two distinct families of thymidylate synthases, ThyA (EC 2.1.1.45) and ThyX [EC 2.1.1.148, (also known as flavin dependent thymidylate synthase FDTS)], without detectable sequence or structural similarity (S. Graziani et al., J Biol Chem 281, 24048 (Aug. 18, 2006); Mathews, II et al., Structure 11, 677 (June, 2003); A. G. Murzin, Science 297, 61 (Jul. 5, 2002); and P. Sampathkumar et al., J Mol Biol 352, 1091 (Oct. 7, 2005)). Whereas human and bacterial ThyA proteins use tetrahydrofolate (H₄folate) to reduce the methylene moiety after its transfer to the uracil ring, ThyX proteins have been proposed to transfer a hydride from the FADH2 co-factor directly to the uracil ring, in the absence of CH₂H₄folate. However, as earlier studies have demonstrated that both substrates, dUMP and CH₂H₄folate, are required for oxidizing ThyX-bound FADH2, the direct proof for the role of dUMP as an initial electron sink is missing.

Because ThyX proteins are essential in many pathogenic bacteria (e.g. Helicobacter, Mycobacteria, Chlamydia and Rickettsia species as well as Bacillus anthracis), they provide a valuable anti-microbial target.

There is a need in the art for the development of new antimicrobial compounds.

The aim of the present invention is to provide new antimicrobial compounds which inhibit specifically ThyX proteins.

The aim of the present invention is to provide new antimicrobial compounds which do not inhibit ThyA proteins.

The present invention relates to a compound having formula (I):

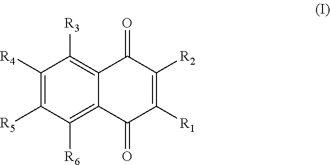

wherein:
R₁ is chosen from the group consisting of:
   phenyl group, possibly substituted,
   —CH₂—C₆H₄—R′₁ group, R′₁ being chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group, R′₁ being preferably in para position, and
   —CH₂—CO—R′ group, R′ representing an aryl or heteroaryl group, said aryl and heteroaryl groups being possibly substituted,
R₂ is chosen from the group consisting of: —OH and halogen,
R₃, R₄, R₅ and R₆ are chosen, independently from each other, in the group consisting of: H, —OH, halogen, alkyl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above;
   or its pharmaceutically acceptable salts, hydrates or hydrated salts or its polymorphic crystalline structures, racemates, diastereomers or enantiomers,
   for its use for the prevention and/or the treatment of bacterial infections.

A particular group of compounds are of formula (I′):

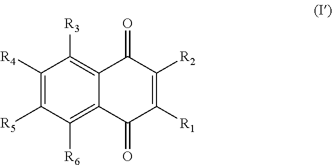

wherein:
R₁ is chosen from the group consisting of: H, cycloalkyl, aryl, —CH₂-aryl, and —CH₂—CO—R′ groups, R′ representing an aryl or heteroaryl group, said cycloalkyl, aryl and heteroaryl groups being possibly substituted,
R₂ is chosen from the group consisting of: —OH and halogen,
R₃, R₄, R₅ and R₆ are chosen, independently from each other, in the group consisting of: H, —OH, halogen, alkyl, —CHO, —CN, —NO₂, —SR_{α}, OR_{α}, —NR_{α}R_{β}, —CONR_{α}R_{β}, —COOR_{α}, and —NHCOR_{α}, R_{α} and R_{β} representing independently from each other H, an alkyl group or an aryl group.

The term “aryl” refers to an aromatic monocyclic, bicyclic, or tricyclic hydrocarbon ring system, wherein any ring atom capable of substitution may be substituted by a substituent. Examples of aryl moieties include, but are not limited to, phenyl, naphthyl, and anthracenyl.

The term “aryl” refers to a group comprising from 6 to 30, preferably from 6 to 20, and for example from 6 to 10 carbon atoms.

The radical —C₆H₄— refers to a divalent phenylene radical. The phenylene ring of radical —C₆H₄— can be either substituted in ortho, meta or para position, and can be represented by the formula:

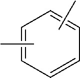

which encompasses the following rings:

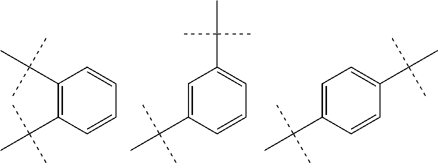

In the present application, preferred aryl groups are phenyl and substituted phenyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution may be substituted by a substituent.

In the present application, preferred heteroaryl groups are aromatic monocyclic compounds comprising 5 or 6 atoms, and most preferably comprising 5 atoms. Preferably, said 5 or 6 membered rings comprise 1 or 2 heteroatoms selected from O, N, or S.

The term “cycloalkyl” as employed herein includes saturated cyclic, bicyclic, tricyclic, or polycyclic hydrocarbon groups having 3 to 12 carbons, wherein any ring atom capable of substitution may be substituted by a substituent. Examples of cycloalkyl moieties include, but are not limited to, cyclohexyl and adamantyl.

In the present application, preferred cycloalkyl groups are cyclohexyl groups, and substituted cyclohexyl groups.

The term “substituents” refers to a group “substituted” on an aryl, heteroaryl or cycloalkyl group at any atom of that group. Suitable substituents include, without limitation, alkyl, alkenyl, alkynyl, aryl, alkoxy, halo, hydroxy, cyano, nitro, amino, —SO₃H, sulphate, phosphate, perfluoroalkyl, perfluoroalkoxy, methylenedioxy, ethylenedioxy, carboxyl, oxo, thioxo, imino (alkyl, aryl, aralkyl), —S(O)ₙ alkyl (where n is 0-2), —S(O)ₙ aryl (where n is 0-2), —S(O)ₙ heteroaryl (where n is 0-2), —S(O)ₙ heterocyclyl (where n is 0-2), amine (mono-, di-, alkyl, cycloalkyl, aralkyl, heteroaralkyl, and combinations thereof), ester (alkyl, aralkyl, heteroaralkyl), amide (mono-, di-, alkyl, aralkyl, heteroaralkyl, and combinations thereof), sulfonamide (mono-, di-, alkyl, aralkyl, heteroaralkyl, and combinations thereof), unsubstituted aryl, unsubstituted heteroaryl, unsubstituted heterocyclyl, and unsubstituted cycloalkyl.

The preferred substituents on aryl or heteroaryl groups are alkyl, amino, amine, hydroxy, alkoxy, halo, perfluoroalkyl such as —CF₃, heterocyclyl, amide, and ester.

The preferred substituents on cycloalkyl, in particular cyclohexyl, groups are aryl groups, and most preferably substituted aryl groups. Most preferred substituents on cyclohexyl groups are aryl groups substituted by at least one halogen atom, and preferably by chlorine atom in para position.

The term “alkyl” means a saturated or unsaturated aliphatic hydrocarbon group which may be straight or branched having about 1 to about 12 carbon atoms in the chain. Preferred alkyl groups have 1 to about 6 carbon atoms in the chain. “Branched” means that one or lower alkyl groups such as methyl, ethyl or propyl are attached to a linear alkyl chain. <<Lower alkyl>> means about 1 to about 4 carbon atoms in the chain which may be straight or branched. The alkyl may be substituted with one or more <<alkyl group substituants>>, which may be the same or different, and include for instance halo, cycloalkyl, hydroxy, alkoxy, amino, acylamino, aroylamino, carboxy.

The terms “arylalkyl” or “aralkyl” refer to an alkyl moiety in which an alkyl hydrogen atom is replaced by an aryl group (possibly substituted). Examples of “arylalkyl” or “aralkyl” include benzyl and 9-fluorenyl groups.

The term “alkoxy” refers to an —O-alkyl radical.

The term “halo” (or “Hal”) refers to the atoms of the group 17 of the periodic table (halogens) and includes in particular fluorine, chlorine, bromine, and iodine atom.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein any ring atom capable of substitution may be substituted by a substituent.

The term “oxo” refers to an oxygen atom, which forms a carbonyl when attached to carbon, an N-oxide when attached to nitrogen, and a sulfoxide or sulfone when attached to sulfur.

The term “alkenyl” as employed herein includes partially unsaturated, nonaromatic, hydrocarbon groups having 2 to 12 carbons, preferably 2 to 6 carbons.

The term “alkynyl” as employed herein includes unsaturated, nonaromatic, hydrocarbon groups having 2 to 12 carbons, preferably 2 to 6 carbons, and comprising at least one triple bond.

The term “acyl” refers to an alkylcarbonyl, cycloalkylcarbonyl, arylcarbonyl, heterocyclylcarbonyl, or heteroarylcarbonyl substituent, any of which may be further substituted by substituents.

As (hetero)aryl groups, the followings may be mentioned:

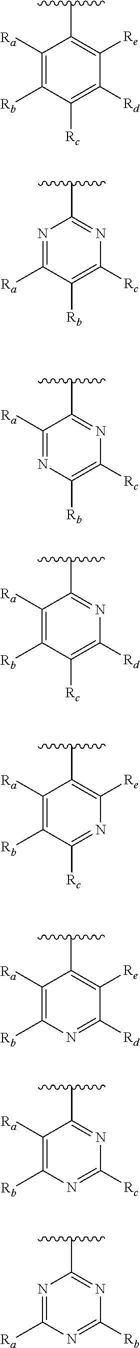

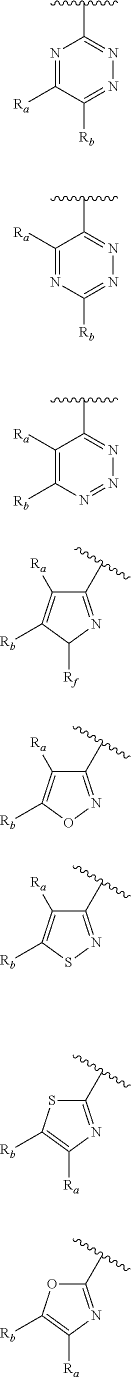

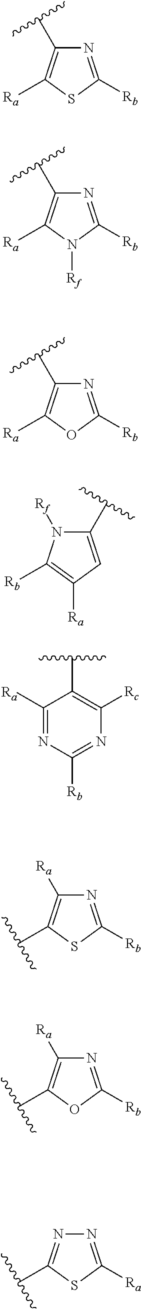

R_{f} being H or an alkyl group,
R_{α}, R_{b}, R_{g}, R_{d}, Rₑ, R_{g}, Rₕ and Rᵢ being chosen, independently from each other, in the group consisting of the following substituents:
   H,
   halogen, such as I, Br, Cl or F,
   alkyl group, said alkyl group being possibly substituted in particular by one or more substituents chosen in the group consisting of the following substituents: halogen, alkenyl or alkynyl groups, aryl groups, —COR_{α}, —COOR_{α}, —SR_{α}, —OR_{α} or —NR_{α}R_{β} groups, R_{α} and R_{β} being as defined above,
   —CHO,
   —CN,
   —NO₂,
   phenyl,
   (hetero)aryl or heterocyclyl, possibly substituted,
   —SR_{α}, —OR_{α}, —NR_{α}R_{β}, —CONR_{α}R_{β}, and —NHCOR_{α}, R_{α} and R_{β} being as defined above.

The compounds herein described may have asymmetric centers. Compounds of the present invention containing an asymmetrically substituted atom may be isolated in optically active or racemic forms. It is well-known in the art how to prepare optically active forms, such as by resolution of racemic forms or by synthesis from optically active starting materials. All chiral, diastereomeric, racemic forms and all geometric isomeric forms of a compound are intended, unless the stereochemistry or the isomeric form is specifically indicated.

The term “pharmaceutically acceptable salt” refers to salts which retain the biological effectiveness and properties of the compounds of the invention and which is are not biologically or otherwise undesirable. In many cases, the compounds of the invention are capable of forming acid and/or base salts by virtue of the presence of amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts see Berge, et al. ((1977) J. Pharm. Sd, vol. 66, 1). The expression “non-toxic pharmaceutically acceptable salts” refers to non-toxic salts formed with nontoxic, pharmaceutically acceptable inorganic or organic acids or inorganic or organic bases. For example, the salts include those derived from inorganic acids such as hydrochloric, hydrobromic, sulfuric, sulfamic, phosphoric, nitric, and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, fumaric, methanesulfonic, and toluenesulfonic acid and the like.

In the context of the invention, the term “treating” or “treatment”, as used herein, means reversing, alleviating, inhibiting the progress of, or preventing the disorder or condition to which such term applies, or one or more symptoms of such disorder or condition.

While it is possible for the compounds of the invention having formula (I) to be administered alone it is preferred to present them as pharmaceutical compositions. The pharmaceutical compositions, both for veterinary and for human use, useful according to the present invention comprise at least one compound having formula (I) as above defined, together with one or more pharmaceutically acceptable carriers and optionally other therapeutic ingredients.

In certain preferred embodiments, active ingredients necessary in combination therapy may be combined in a single pharmaceutical composition for simultaneous administration.

As used herein, the term “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions; however, solid forms suitable for solution, or suspensions, in liquid prior to use can also be prepared. The preparation can also be emulsified. In particular, the pharmaceutical compositions may be formulated in solid dosage form, for example capsules, tablets, pills, powders, dragees or granules.

The choice of vehicle and the content of active substance in the vehicle are generally determined in accordance with the solubility and chemical properties of the active compound, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

The pharmaceutical compositions can be administered in a suitable formulation to humans and animals by topical or systemic administration, including oral, rectal, nasal, buccal, ocular, sublingual, transdermal, rectal, topical, vaginal, parenteral (including subcutaneous, intra-arterial, intramuscular, intravenous, intradermal, intrathecal and epidural), intracisternal and intraperitoneal. It will be appreciated that the preferred route may vary with for example the condition of the recipient.

The formulations can be prepared in unit dosage form by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Total daily dose of the compounds of the invention administered to a subject in single or divided doses may be in amounts, for example, of from about 0.001 to about 100 mg/kg body weight daily and preferably 0.01 to 10 mg/kg/day. Dosage unit compositions may contain such amounts of such submultiples thereof as may be used to make up the daily dose. It will be understood, however, that the specific dose level for any particular patient will depend upon a variety of factors including is the body weight, general health, sex, diet, time and route of administration, rates of absorption and excretion, combination with other drugs and the severity of the particular disease being treated.

A particular group of compounds of the present invention consists of compounds of formula (I) wherein R₂ is halogen, in particular Br.

Such compounds may be represented by the below formula (I-1):

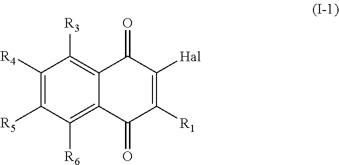

Hal representing a halogen atom, in particular Br.

Preferably, in formula (I-1), R₄, R₅ and R₆ are H.

A particular group of compounds of formula (I′) consists of compounds of formula (I′) wherein R₁ is H and R₂ is halogen, in particular Br.

Such compounds may be represented by the below formula (I-1′):

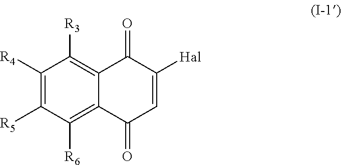

Hal representing a halogen atom, in particular Br.

Preferably, in formula (I-1′), R₄, R₅ and R₆ are H.

Another particular group of compounds of the present invention consists of compounds of formula (I-1) wherein R₄, R₅ and R₆ are H and Hal is Br.

Such compounds may be represented by the below formula (I-2):

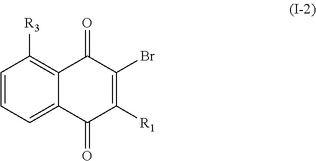

wherein R₃ is chosen in the group consisting of: H, —OH, halogen, alkyl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group.

Preferably, in formula (I-2), R₃ is —OH.

Another particular group of compounds of the present invention consists of compounds of formula (I) wherein R₂ is —OH.

Such compounds may be represented by the below formula (I-3):

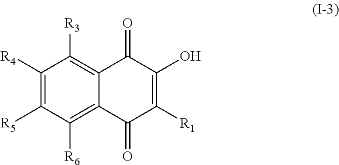

Preferably, in formula (I-3), R₃ is —OH.

Another particular group of compounds of formula (I-1′) consists of compounds of formula (I-1′) wherein R₄, R₅ and R₆ are H and Hal is Br.

Such compounds may be represented by the below formula (I-2′):

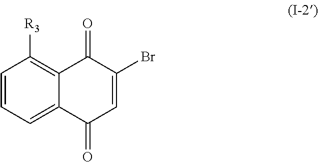

wherein R₃ is chosen in the group consisting of: H, —OH, halogen, alkyl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group.

Preferably, in formula (I-2′), R₃ is —OH.

A particular group of compounds of the present invention consists of compounds of formula (I) wherein R₃ is —OH and R₄, R₅ and R₆ are H.

A particular group of compounds of formula (I′) consists of compounds of formula (I) wherein R₃, R₄, R₅ and R₆ are H, R₂ is —OH, and R₁ is a cycloalkyl group, preferably a substituted cyclohexyl group.

A preferred compound of this group is atovaquone (CAS RN 95233-18-4) and has the following formula:

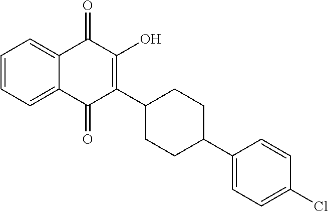 [SMILES: O=C1C(O)=C(C2CCC(c3ccc(Cl)cc3)CC2)C(=O)c2ccccc21]

A group of compounds of the present invention consists of compounds of formula (I) wherein R₃, R₄, R₅ and R₆ are H, and R₂ is —OH.

According to an embodiment, the present invention relates to compounds having formula (II):

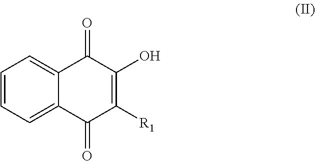

wherein R₁ is as defined above in formula (I), for their use for the prevention and/or the treatment of bacterial infections.

According to a particular embodiment, compounds of formula (I′) are of formula (II′):

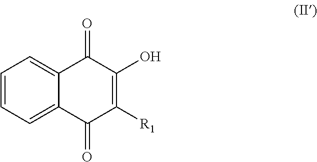

wherein R₁ is chosen from the group consisting of: aryl, —CH₂-aryl, and —CH₂—CO—R′ groups, R′ being as defined above in formula (I′), for their use for the prevention and/or the treatment of bacterial infections.

According to a particular embodiment, the present invention relates to compounds having formula (II) wherein R₁ is a phenyl group, possibly substituted, for their use for the prevention and/or the treatment of bacterial infections.

When R₁ is a substituted phenyl group, it may comprise one or several substituents, and in particular one or two substituents.

When R₁ is a substituted phenyl group, the substituents are chosen from the group consisting of: —CHO, —CN, aryl, alkyl, halo, nitro, hydroxy, —SR_{α}, —OR_{α}, —NR_{α}R_{β}, —CONR_{α}R_{β}, —COOR_{α}, and —NHCOR_{α}, R_{α} and R_{β} being as defined above.

According to a preferred embodiment, R₁ is a phenyl group with one substituent in para position.

According to a particular embodiment, in formula (II) as defined above, R₁ is a phenyl group, substituted by an aryl group, and preferably a phenyl group.

As preferred compounds of the invention, one may refer to a compound having formula (II) wherein R₁ is a biphenyl group.

According to an advantageous embodiment, in the above formula (II), R₁ is chosen from the group consisting of: —CH₂-aryl and —CH₂—CO—R′ groups, R′ being as defined above.

Preferably, when R₁ is a —CH₂-aryl group, said aryl group is a phenyl group or a substituted phenyl group. When said aryl group is a substituted phenyl group, it may comprise one or several substituents, and in particular one or two substituents.

According to an advantageous embodiment, in the above formula (II), R₁ is chosen from the group consisting of: —CH₂—C₆H₄—R′₁ group and —CH₂—CO—R′ group, R′₁ and R′ being as defined above.

According to an advantageous embodiment, in the above formula (I), R₁ is chosen from the group consisting of: —CH₂—C₆H₄—R′₁ group and —CH₂—CO—R′ group, R′₁ and R′ being as defined above.

The present invention thus also relates to compounds for the use as defined above, having formula (I) wherein R₁ is chosen from the groups having the following formula (A):

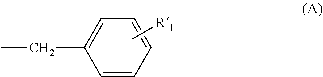

R′₁ being as defined above.

In formula (A) above, the phenyl group may comprise several substituents, that is to say it may comprise several R′₁ groups being identical or different.

According to a particular embodiment, in formula (A) above, R′₁ is a group in para position having formula —OR′₂, R′₂ being H or an alkyl group comprising from 1 to 6 carbon atoms, and preferably from 1 to 3 carbon atoms.

Most preferably, R′₁ is a para-methoxy group.

Another group of compounds of the invention is constituted by compounds having formula (II) wherein R₁ is chosen from the groups having formula —CH₂—CO—R′, R′ being as defined above.

Another group of compounds of the invention is constituted by compounds having formula (I) wherein R₁ is chosen from the groups having formula —CH₂—CO—R′, R′ being as defined above.

Preferably, R′ is chosen from the aryl groups, possibly substituted.

Most preferably, R′ is a phenyl group, possibly substituted.

When R₁ is a substituted phenyl or aryl group, it may comprise one or several substituents, and in particular one or two substituents.

When R₁ is a substituted phenyl or aryl group, the substituents are chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above.

According to a preferred embodiment, R′ is a phenyl group with one substituent in para position.

The present invention thus also relates to compounds for the use as defined above, having formula (II) wherein R₁ is chosen from the groups having formula (B):

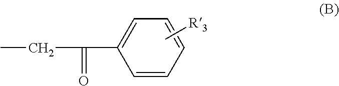

R′₃ being chosen from substituents chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above, and R′₃ being preferably in para position.

The present invention thus also relates to compounds for the use as defined above, having formula (I) wherein R₁ is chosen from the groups having formula (B):

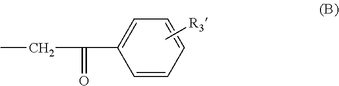

R′₃ being chosen from substituents chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above, and R′₃ being preferably in para position.

In formula (B) above, the phenyl group may comprise several substituents, that is to say it may comprise several R′₃ groups being identical or different.

According to a particular embodiment, in formula (B) above, R′₃ is a group in para position chosen from the alkyl groups comprising from 1 to 6 carbon atoms, and preferably from 1 to 3 carbon atoms, and the groups having formula —OR′₄, R′₄ being H or an alkyl group comprising from 1 to 6 carbon atoms, and preferably from 1 to 3 carbon atoms.

Most preferably, R′₃ is a para-methoxy group or a para-methyl group.

Another group of compounds of the invention is constituted by compounds having formula (II) wherein R₁ is chosen from the groups having formula —CH₂—CO—R′, R′ being chosen from the heteroaryl groups, possibly substituted.

Another group of compounds of the invention is constituted by compounds is having formula (I) wherein R₁ is chosen from the groups having formula —CH₂—CO—R′, R′ being chosen from the heteroaryl groups, possibly substituted.

The present invention thus also relates to compounds for the use as defined above, having formula (II) wherein R₁ is chosen from the groups having formula (C):

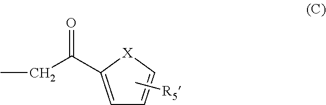

R′₅ being chosen from H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above, and X being chosen from the group consisting of: S, O, and NH, X being preferably S.

The present invention thus also relates to compounds for the use as defined above, having formula (I) wherein R₁ is chosen from the groups having formula (C):

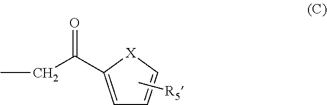

R′₅ being chosen from H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined above, and X being chosen from the group consisting of: S, O, and NH, X being preferably S.

In formula (C) above, the phenyl group may comprise several substituents, that is to say it may comprise several R′₅ groups being identical or different.

The most preferred compounds of the invention are the following compounds:

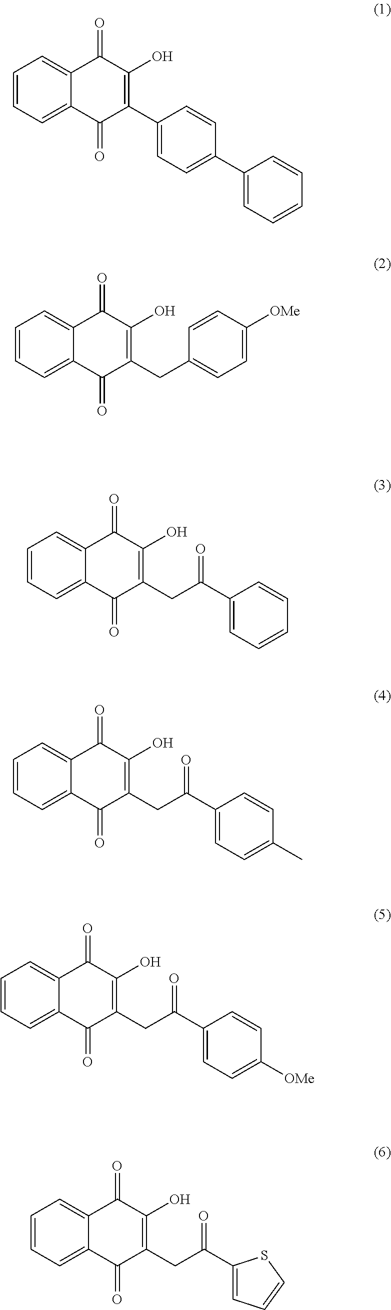 [SMILES: O=C1C(O)=C(c2ccc(-c3ccccc3)cc2)C(=O)c2ccccc21 ; COc1ccc(CC2=C(O)C(=O)c3ccccc3C2=O)cc1 ; O=C(CC1=C(O)C(=O)c2ccccc2C1=O)c1ccccc1 ; Cc1ccc(C(=O)CC2=C(O)C(=O)c3ccccc3C2=O)cc1 ; COc1ccc(C(=O)CC2=C(O)C(=O)c3ccccc3C2=O)cc1 ; O=C(CC1=C(O)C(=O)c2ccccc2C1=O)c1cccs1]

Preferably, the present invention relates to the compounds as defined above for their use for the prevention and/or the treatment of bacterial infections chosen from Mycobacterium or Helicobacter species bacteria infections, and preferably from Mycobacterium smegmatis and Helicobacter pylori infections.

Preferably, the present invention relates to the compounds as defined above for their use for the prevention and/or the treatment of bacterial infections, due to bacteria expressing ThyX protein.

Among bacteria expressing ThyX protein, one may cite Borrelia burgdorferi, Campylobacter jejuni, Chlamydia trachomatis, Chlamydia pneumoniae, Clostridium botulinum, Clostridium difficile, Clostridium tetani, Corynebacterium diphteriae, Helicobacter pylori, Leptospira interrogans, Mycobacterium leprae, Mycobacterium tuberculosis, Mycobacterium bovis, Rickettsia prowazeki, Rickettsia rickettsii, and Treponema pallidum.

The present invention relates to compounds having formula (I) as defined above for their use for the prevention and/or the treatment of bacterial infections by inhibition of ThyX protein.

The compounds of formula (I) of the present invention are specific ThyX inhibitors and they are compounds known from the skilled person. In particular, above compounds (1), (2), (3), (4), (5) and (6) have the following CAS RN 67287-16-5, 59304-99-3, 59382-19-3, 412966-59-7, 413571-44-5, and 722461-11-2, respectively.

### FIGURES

FIG. 1 represents dose-response curves for compounds of the invention is showing the fractional velocity of the reaction as function of inhibitor concentration are shown. IC₅₀ values were derived by fitting the data to sigmoidal dose-response equation.

FIG. 2 represents the initial velocity of NADPH oxidation as a function of dUMP concentration in absence or presence of atovaquone. Atovaquone concentrations in the assays are indicated on the right.

FIG. 3 represents the Lineweaver-Burk plot in absence or presence of atovaquone. Atovaquone concentrations in the assays are indicated on the right.

### EXAMPLES

With the goal to identify ThyX-specific inhibitors, the National Cancer Institute diversity set (1982 molecules) and 340 natural compounds (Greenpharma) using the Paramecium bursaria chlorella virus (PBCV-1) enzyme have been screened (F. Esra Onen et al., Bioorg Med Chem Lett 18, 3628 (Jun. 15, 2008)).

Medium Throughput Primary Screen to Identify ThyX Inhibitors

The NADPH oxidation assay for PBCV-1 ThyX activity (Graziani, S., Xia, Y., Gurnon, J. R., Van Etten, J. L., Leduc, D., Skouloubris, S., Myllykallio, H., and Liebl, U. (2004) Functional analysis of FAD-dependent thymidylate synthase ThyX from Paramecium bursaria Chlorella virus-1. J. Biol. Chem. 279, 54340-54347) was adapted for a medium throughput inhibitor screen (MTS) in 96-well plates. A typical assay with a final volume of 200 μl, contained 200 μM NADPH, 2 μM CH2H4folate, 5 μM dUMP, 1 mM MgCl2, 1% glycerol, 62.5 μM FAD and 400 nM PBCV-1 ThyX. 1982 compounds from the National Cancer Institute diversity set 1 and 340 natural compounds, selected for their diversity and drug-likeness (GreenPharma) (all in DMSO) were added to a final concentration of 20 μM for the initial screen. The microplates were prepared by a liquid handling robotic workstation (Xiril X75), and manually transferred to the automated microplate reader Chameleon II (Hidex). The reactions were started by automatically injecting NADPH into individual wells and the decrease in absorbance at 340 nm was followed at 37° C. for 5 min. Samples with added DMSO and no enzyme were used as positive and negative controls, respectively. For NADPH oxidation at 340 nm a molar extinction coefficient of 6220 M⁻¹ cm⁻¹ was used. For this assay a Z′-factor value of 0.62 was calculated, is indicating robustness of the measurement and an acceptable dynamic range of the signal (Zhang, J. H., Chung, T. D., and Oldenburg, K. R. (1999) J. Biomol. Screen. 4, 67-73). Primary hits were defined as compounds that inhibited 75% of ThyX activity at the initial screening concentration.

Secondary Screen and Lead Selection

First round screening hits were tested on C. trachomatis and M. tuberculosis ThyX using the NADPH oxidation assay described above. The activity of H. pylori ThyX was determined using a tritium release assay that measures deprotonation at the 5-position of the pyrimidine ring of dUMP substrate and detects the formation of tritiated water during the ThyX reaction (Leduc, D., Graziani, S., Lipowski, G., Marchand, C., Le Marechal, P., Liebl, U., and Myllykallio, H. (2004) Proc. Natl. Acad. Sci. U.S.A. 101, 7252-7257).

This assay was adapted for automatization to a 96-well plate format. A typical reaction mix contained 50 mM HEPES pH 7.5, 1 mM MgCl₂, 10% glycerol, 62.5 μM FAD, 2.5 μM dUMP, 200 μM NADPH, 200 μM CH₂H₄folate (Merck Eprova) and 400 nM of ThyX in a in final volume of 25 μL. The specific activity of the [5-H³]dUMP stock (Moravek) used was 13.6 Ci/mmol. The reactions were started by adding the enzyme, and the assay mixtures were incubated for 15 minutes at 37° C. The reaction was then stopped by addition of 175 μL of 10% (wt/vol) activated Norit A charcoal (Sigma) in 2% trichloroacetic acid to remove radioactive nucleotides from the reaction mixture. Released tritium was separated from the reaction substrates using vacuum manifold and Unifilter 350 GF/C plates (Whatman) during 15 minutes at −20 mm Hg. The radioactivity remaining in the filtrate was measured by adding Ecolune (ICN) scintillation liquid and counted with the Chameleon II microtiterplate reader. All reactions were done in triplicate and included controls without compounds, without enzyme and with DMSO only.

The effect of the primary hits on the activity of human ThyA was determined by following the increase in absorbance at 340 nm that accompanies the conversion of CH₂H₄folate into H₂folate during the ThyA catalyzed reaction (Meek T D, Garvey E P, Santi D V. Purification and characterization of the bifunctional thymidylate synthetase-dihydrofolate reductase from methotrexate-resistant Leishmania tropica. Biochemistry. 1985 Jan. 29; 24(3):678-86). Measurements were done in 96-well microplates with the typical assay containing 50 mM HEPES pH 8, 1 mM MgCl₂, 1% glycerol, 150 μM dUMP, 100 μM CH2H4folate, 430 nM human ThyA in 200 μL final volume. Primary hits were included in reaction mixtures at a concentration of 20 μM. The reaction was followed for 15 min at 37° C. All reactions were done in triplicate and included no compound, DMSO and no enzyme controls. A molar extinction coefficient of 12,300 M⁻¹ cm⁻¹ for H₂folate at 340 nm was used for calculation of the specific activity (Hillcoat B L, Nixon P F, Blakley R L. Effect of substrate decomposition on the spectrophotometric assay of dihydrofolate reductase. Anal Biochem. 1967; 21:178-189). Non-specific inhibition by aggregation or non-specific protein binding were tested by addition of 0.1% of Triton X-100 or 0.1 mg/ml of bovine serum albumine (BSA) to the assays, respectively.

Dedicated and Focused Library Screen

160 1,4-naphtoquinone (1,4 NQ) derivatives were purchased from Chembridge Corporation along with a control library that consisted of 80 1,2 NQ derivatives. Isis and Chime (MDL) were used to select for molecules with the following criteria: molecular weight between 160 and 480 Da, number of Hdonors less than 5 and number of H-acceptors less than 10, calculated XlogP value between 0 and 5.6, total polar surface area less than 150 A² and number of rotative bonds between 2 and 10.

Compounds that inhibited PBCV-1 ThyX at 20 μM concentration in the NADPH oxidation assay were selected as hits. For each hit IC50 values were determined by fitting the data from three independent experiments to the equation for a dose-response sigmoid curve using GraphPad Prism 4 Software (GraphPad Software Inc.). The library consisting of 522-hydroxy 1,4-naphthoquinones was constructed using molecules purchased from Chembridge Corporation or Sigma.

To increase robustness and specificity of ThyX inhibitors, additional assays have been performed. First, the effects of fourteen ThyX inhibitors have been tested using NADPH oxidation assays or by following the loss of tritium from [5-³H]dUMP in a microtiter plate-4 compatible format. The use of two individual assays that measure the early and late reaction steps of ThyX catalysis excluded the possibility that the results obtained using the colorimetric assay were affected by the absorption properties of the screened molecules. These tests on ThyX proteins have been performed from Helicobacter pylori, Chlamydia trachomatis, and Mycobacterium tuberculosis, and used human thymidylate synthase ThyA as the negative control. These tests identified a single compound that drastically decreased ThyX activity in the two independent enzymatic assays, but did not influence the activity of human thymidylate synthase ThyA

These assays have led to the identification of the compound 2E04:

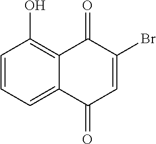 [SMILES: O=C1C=C(Br)C(=O)c2c(O)cccc21]

The identified compound, 2-bromo-8-hydroxy-1,4-naphthoquinone (compound 2E04) was originally isolated from the fruit of malaysian persimmon (Diospyros maritima), and is related to the lipid-soluble vitamin K1 [phylloquinone (also of plant origin)] that is required for efficient blood coagulation.

We found that the molecule 2E04 at a concentration of 1.5 μg/ml (≈5 μM) reproducibly inhibited in thymidine-deprived medium the growth of an E. coli strain where PBCV-1 thyX replaces the chromosomal copy of thyA [E. coli FE010 (ΔthyA::thyX)](F. Escartin, S. Skouloubris, U. Liebl, H. Myllykallio, Proc Natl Aced Sci USA 105, 9948 (Jul. 22, 2008)).

This bactericidal activity was not observed with an isogenic wild type strain prompting a design of a focused library containing 160 commercially available 1,4-naphthoquinones (1,4-NQ), together with a control library of 80 1,2-naphthoquinones (Chembridge).

The 1,4-NQ library provided eight additional ThyX inhibitors, whereas no inhibitors were identified using the control library. The below table 1 indicates the chemical structures of the obtained hits.

TABLE 1
Hits issued from the screen of the library of 160 1,4-naphthoquinone
derivatives. Percentage of inhibition using deprotonation assays in the presence of
20 μM compounds is indicated.

| | | % inhibition |
|---|---|---|
| | | (deprotonation) |
| Compound | Structure | PBCV-1 ThyX |
| C8-C1 2-hydroxy-3-(4- methoxybenzyl)-1,4- naphthoquinone | 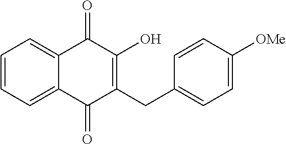 [SMILES: COc1ccc(CC2=C(O)C(=O)c3ccccc3C2=O)cc1] | 78 ± 4 |
| F6-C1 2-(biphenyl-4-yl)-3- hydroxy-1,4- naphthoquinone | 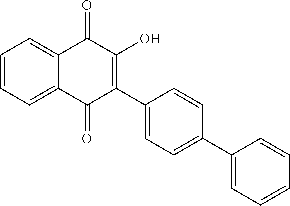 [SMILES: O=C1C(O)=C(c2ccc(-c3ccccc3)cc2)C(=O)c2ccccc21] | 95 ± 3 |
| E11-C1 2-hydroxy-3-(2-oxo-2- phenylethyl)-1,4- naphthoquinone | 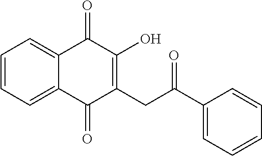 [SMILES: O=C(CC1=C(O)C(=O)c2ccccc2C1=O)c1ccccc1] | 36 ± 5 |
| H3-C1 2-hydroxy-3-(2-oxo-2- (thiophen-2-yl)ethyl)-1,4- naphthoquinone | 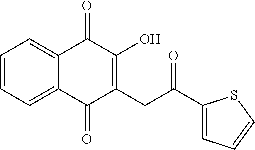 [SMILES: O=C(CC1=C(O)C(=O)c2ccccc2C1=O)c1cccs1] | 38 ± 3 |
| F3-C1 2-hydroxy-3-(2-oxo-2-p- tolylethyl)-1,4- naphthoquinone | 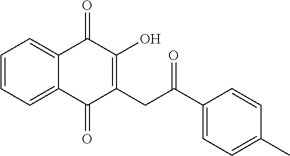 [SMILES: Cc1ccc(C(=O)CC2=C(O)C(=O)c3ccccc3C2=O)cc1] | 38 ± 7 |
| G3-C1 2-hydroxy-3-(2-(4- methoxyphenyl)-2- oxoethyl)-1,4- naphthoquinone | 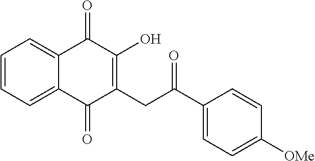 [SMILES: COc1ccc(C(=O)CC2=C(O)C(=O)c3ccccc3C2=O)cc1] | 22 ± 7 |
| B3-C1 2-(5-hydroxy-1,4-dioxo-1,4- dihydro-naphthalen-2- ylamino)benzoic acid | 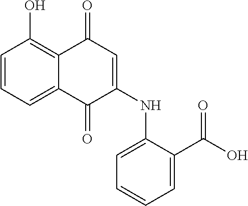 [SMILES: O=C(O)c1ccccc1NC1=CC(=O)c2c(O)cccc2C1=O] | 12 ± 6 |

Notably, despite the chemical diversity of the screened molecules, 1,4-NQ derivatives carrying a hydroxyl group at position 2 were frequently identified, revealing 2-hydroxyl-1,4-NQs as biologically active ThyX inhibitors.

To provide proof of concept for this notion, 52 additional 2-hydroxyl-1,4-NQs carrying different chemical moieties at position 3 have been tested. These to experiments revealed that 29% of the compounds from the third generation library acted as ThyX inhibitors at 20 μM concentration and that the most active molecules predominantly carried aromatic ring groups at the 3 position. On the other hand, more 5 bulky groups, such as long alkyl chains or branched aryl groups diminished the inhibitory potential.

IC50 Determination Using Dose-Response Curves

All tests were performed in triplicates in 96 well microtiter plates. A typical assay (200 μL) contained 200 μM NADPH, 2 μM dUMP, 1 mM MgCl₂, 1% glycerol, 10 μM FAD, 200 nM of PBCV-1 ThyX and different concentrations of inhibitors ranging from 0.1 μM to 40 μM. The reactions were started by injection of NADPH and the absorbance at 340 nm was followed for 3 min at 37° C. DMSO and no enzyme were used as high and no activity controls. A molar extinction coefficient for NADPH oxidation at 340 nm of 6220 M⁻¹ cm⁻¹ was used for quantification of absorption changes. The optical path length was 0.5 cm. The obtained Vᵢ/V₀ values were plotted for each inhibitor concentration and fitted to sigmoidal dose-response curves using GraphPad Prism 4 Software.

Bacterial Strains

Helicobacter pylori strain 26695 and SS1 were grown on either blood agar base two (Oxoid) plates supplemented with 10% defibrinated horse blood (Oxoid) or in liquid culture in brain-heart infusion broth (Oxoid) supplemented with 10% FBS (Gibco BRL). An antibiotics-fungicide mix consisting of vancomycin (final concentration 10 μg/ml), polymyxin B (2.5 units per liter), trimethoprim (5 μg/ml), and fungizone (2.5 μg/ml) was added. Plates were incubated at 37° C. under microaerobic atmosphere in jars using the CampyGen gas generating system (Oxoid). Liquid cultures were shaken at 175 rpm.

Mycobacterium smegmatis strain mc2 155 was grown on either LB agar plates or in liquid culture in LB medium supplemented with 0.2% glycerol and 0.05% Tween 80 (Sigma). Liquid cultures were shaken at 175 rpm.

Antimicrobial Testing

Cultures of E. coli MG1655 and E. coli FE10 ΔthyA::thyX (Escartin et al., 2008) were grown in LB medium overnight. The cells were then harvested and washed two times with M9 medium to remove the remaining thymidine. The cells were diluted to OD600 of approximately 0.1 in M9 medium or M9 medium supplemented with 40 μg/ml of thymidine. 1 ml cells suspension was transferred to 24 well microplates (Nunc) and 10 μL of 2E04 was then added to a final concentration of 60 μM to half of the wells. No cells and DMSO-only controls were included for each medium composition. The plates were closed with appropriate plastic lids and incubated at 37° C. with shaking at 160 rpm. After 24 h of incubation, 100 μL of 10 fold serial dilutions were plated onto LB plates or LB plates supplemented with kanamycin (40 μg/ml). Colonies were enumerated after overnight incubation at 37°. Colony forming units (CFU) per milliliter for each growth condition were determined from three independent experiments.

Minimal inhibitory concentrations (MICS) were determined using a broth microdilution test in 24 well microtiter plates. For test inoculums an overnight preculture of H. pylori strain 26695 was diluted to OD600 of approximately 0.1 and 1 ml of this suspension was transferred to each well. 10 μL of two-fold serial dilutions of each compound in DMSO were added and the covered plates were incubated for 24 h at 37° C. with orbital shaking at 150 rpm under microaerophilic conditions obtained using CampyGen gas generating system (Oxoid). The MIC was determined as the lowest compound concentration resulting in complete growth inhibition after 24 h of incubation. All the tests were done in triplicates and they included no cells and DMSO-only controls. MIC values for 2-hydroxy-1,4-naphthoquinones were derived from the standard curve based on the diameter of the inhibition zone for H. pylori 26695 measured in disc diffusion tests. The curve was obtained by plotting the diameter of inhibition zone in disc diffusion tests against MIC values of 5 representative compounds. For disc diffusion test an overnight culture of H. pylori strain 26695 was diluted to OD600≈0.01 and spread on agar plates by inundation. 10 μg of each compound were deposited onto diffusion discs and plates were incubated under microaerophilic conditions for 72 h. The diameters of each inhibition zone were measured (disk surface and a DMSO inhibition zone were subtracted). All experiments were done in triplicate, including a DMSO-only control on each plate. MIC determination by broth microdilution was not possible for M. smegmatis mc2 155 due to build up of cell aggregates in liquid cultures. The susceptibility of M. smegmatis mc2 155 towards selected compounds was thus tested using disc diffusion test on LB-agar plates as described above. The inhibition diameters were read after 48 h of incubation at 37° C.

Some compounds having formula (II) as defined above have been tested for their in vitro antimicrobial activity (see below table 2).

TABLE 2
IC50 and MIC of compounds of formula (II)

| Molecule | R₁ | IC₅₀ | MIC |
|---|---|---|---|
| F6-C1 (Chembridge) | 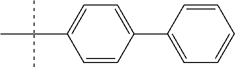 | 200 nM | 10 μg/mL |
| G3-C1 (Chembridge) | 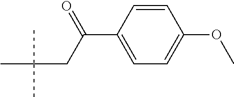 | 13 μM | 10 μg/mL |
| C8-C1 (Chembridge) | 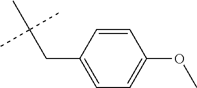 | 5 μM | 2.5-5 μg/mL |
| A4-C2 (Chembridge) | 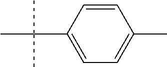 | 9 μM | 4.7 μg/mL |
| H3-C1 (Chembridge) | 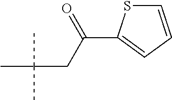 | 8 μM | 10 μg/mL |
| A7-S (Sigma) | 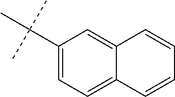 | 1.2 μM | 6 μg/mL |
| A4-S (Sigma) | 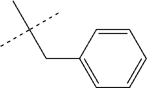 | 1.4 μM | 1.4 μg/mL |
| C8-S (Sigma) | 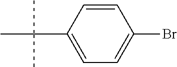 | 1.2 μM | 7 μg/mL |
| A3-C2 (Chembridge) | 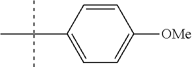 | 8 μM | 4.7 μg/mL |

Anti-Microbial Properties

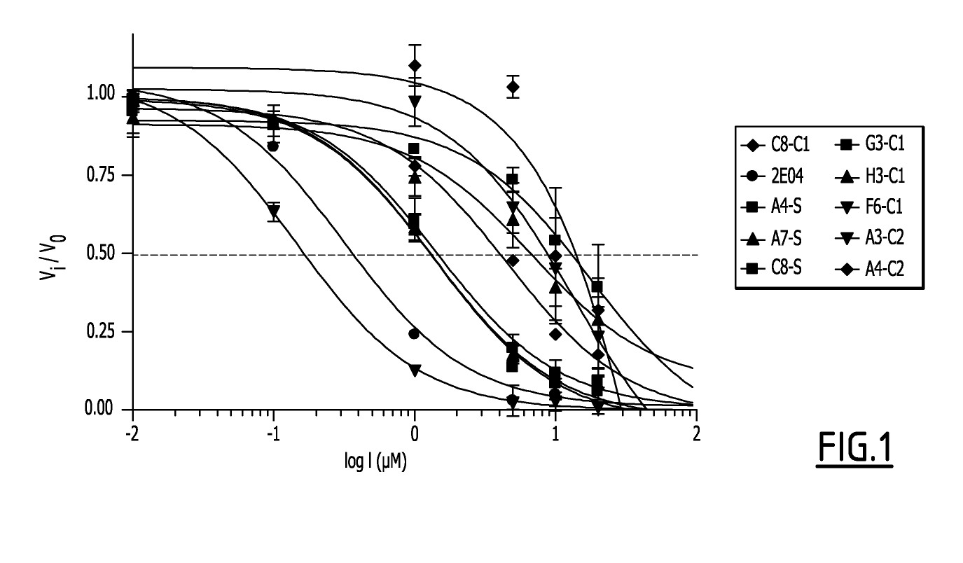

We then investigated the anti-microbial properties of 2-hydroxyl-1,4-NQs acting as ThyX inhibitors against Helicobacter pylori 26695 and Mycobacterium smegmatis mc2155. These experiments established the correlation between the inhibition surface in disc-diffusion tests and MIC values determined from liquid cultures of H. pylori. Disc diffusion tests for M. smegmatis were readable, whereas formation of cell aggregates in liquid cultures of M. smegmatis prevented accurate determination of MIC values. By combining these results with in vitro activities expressed as 1050 values, we identified a subset of 9 compounds as potent ThyX inhibitors with specific antimicrobial activity against Helicobacter pylori 26695 and Mycobacterium smegmatis mc2155, but no activity against thyA-carrying E. coli. FIG. 1 shows the dose-response plots for these compounds that were measured is at fixed substrate concentration. All of these compounds are drug-like and their predicted physicochemical parameters are comparable to atovaquone—one of the active substances of the clinically approved antimalarial drug Malarone®.

The compound C8-C1 [-hydroxy-3-(4-methoxybenzyl)-1,4-naphthoquinone] inhibited the growth of both strains, with a minimal inhibitory concentration (MIC) of ≈2.5 μg/ml for Helicobacter pylori in disc diffusion tests and liquid cultures. The molecule C8-C1 was an obvious choice for more detailed mechanistic and structural studies as we found it to be non-genotoxic and to exhibit low cytotoxic activity, which was further decreased in metabolic activation tests using cultured human lymphoblastoid TK6 cells (P. W. Hastwell et al., Mutagenesis 24, 455 (September, 2009)).

Cytotoxicity and Genotoxicity Tests Using Human Cell Lines

Genotoxicity and cytotoxicity of 2E04 and 5 compounds issued from the screen of the dedicated library of 160 1,4-NQs were determined using the GreenScreen HC genotoxicity screening assay without metabolic activation. Two strains of cultured human lymphoblastoid TK6 cells were used, the test strain (GenM-T01) and the non-fluorescent control strain (GenM-001), the latter was used to allow correction for any autofluorescence from the test. The plates were analyzed at 24 hour and 48 hour time points using a microplate reader, that allows measurements of light absorbance and fluorescence for cells and solutions in the microplate wells. Absorbance is proportional to cell proliferation, which is lowered by toxic analytes, and fluorescence is proportional to the activity of the cell's DNA repair system, which is increased by genotoxic analytes. After a first round of tests, compound C8-C1 was selected for further testing using GreenScreen HC with metabolic activation using the S9 fraction. The GreenScreen HC +S9 protocol uses the same cultured TK6 cell strains (GenM-001 and GenMT01). 10 μg/ml of compound C₈-C₁ was incubated with both cell strains in the presence of 1% v/v S9 fraction mix in Exposure Medium at 37° C. (5% CO₂, 95% humidity) for 3 hours. After a recovery period of 45 hours the GFP fluorescence signal and cell viability (assessed by propidium iodide uptake) were measured.

The absence of a free position in the quinone ring prevents its participation in non-specific arylation reactions (G. Ludewig, S. Dogra, H. Glatt, Environ Health Perspect 82, 223 (July, 1989); P. J. O'Brien, Chem Biol Interact 80, 1 (1991)), presumably lowering cytotoxicity of the compound C8-C1. Overall, these studies established that compound C8-C1 provides an excellent compromise between robust antimicrobial activity and low cytotoxicity.

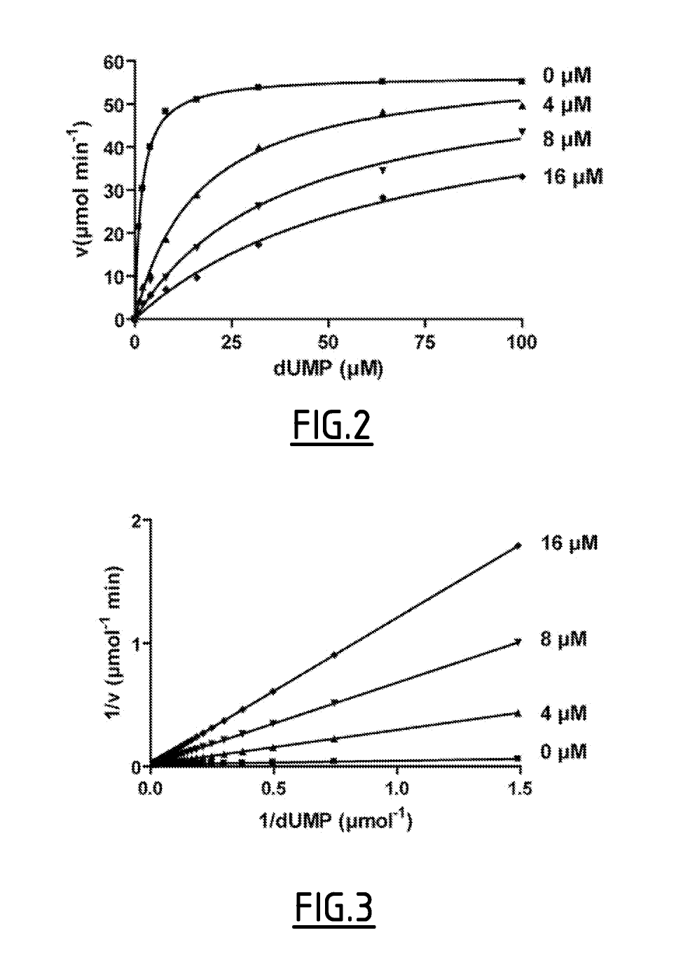

To establish the mechanism of ThyX inhibition, the initial velocities of NADPH oxidation have been measured in the presence of the inhibitors C8-C1, 2EO4, as well as atovaquone by varying dUMP concentrations. These assays indicated that these compounds increased the apparent Km value of the enzyme for dUMP with little effect on the reaction rate at high dUMP concentrations. Fitting of the initial velocities using a competitive inhibition model (goodness of fit value 0.97-0.99) allowed determination of Ki-values of 0.25 μM for C₈-C₁, 1.5 μM for 2E04 and 5 μM for atovaquone (FIG. 2).

Since in bacteria the intracellular dUMP concentration is relatively low (≈10 μM) to prevent uracil incorporation during chromosomal DNA replication, these data indicate that these inhibitors are capable of releasing dUMP from the enzyme in vivo (note also that typical Km, dUMP values for ThyX proteins are 5-15 μM). Notably, in the presence of physiological dUMP concentrations, ThyX inhibition does not depend on the redox activity of naphthoquinones. Consequently, the inhibitors of the invention markedly decelerate the unusual hydride transfer reaction catalyzed by ThyX proteins. This is of importance as the redox activity of 1,4-naphthoquinones also plays a key role in their cytotoxicity.

1. A compound having formula (I): 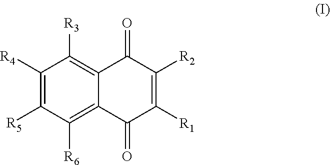
wherein:
R₁ is chosen from the group consisting of:
phenyl group, possibly substituted,
—CH₂—C₆H₄—R′₁ group, R′₁ being chosen from the group consisting of: H, —OH, halogen, alkyl, aryl, CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group, R′₁ being preferably in para position, and
—CH₂—CO—R′ group, R′ representing an aryl or heteroaryl group, said aryl and heteroaryl groups being possibly substituted,
R₂ is chosen from the group consisting of: —OH and halogen,
R₃, R₄, R₅ and R₆ are chosen, independently from each other, in the group consisting of: H, —OH, halogen, alkyl, —CHO, —CN, —NO₂, —OR_{α}, —NR_{α}R_{β}, —CONR_{α}R_{β}, —COOR_{α}, and —NHCOR_{α}, R_{α} and R_{β} being as defined above, or its pharmaceutically acceptable salts, hydrates or hydrated salts or its polymorphic crystalline structures, racemates, diastereomers or enantiomers.

2. The compound according to claim 1, having formula (II): 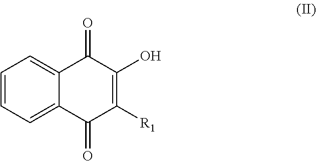
wherein R₁ is as defined in claim 1.

3. The compound according to claim 1, wherein R₁ is a substituted or unsubstituted phenyl group.

4. The compound according to claim 1, wherein R₁ is a phenyl group, substituted by an aryl group.

5. The compound according to claim 1, wherein R₁ is chosen from the group consisting of: —CH₂—C₆H₄—R′₁ group and —CH₂—CO—R′ group, R′₁ and R′ being as defined in claim 1.

6. The compound according to claim 1, wherein R₁ is chosen from the groups having the following formula (A): 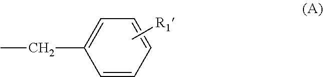
R′₁ being as defined in claim 1.

7. The compound according to claim 6, wherein R′₁ is a group in para position having formula —OR′₂, R′₂ being H or an alkyl group comprising from 1 to 6 carbon atoms.

8. The compound according to claim 1, wherein R₁ is chosen from the groups having formula —CH₂—CO—R′, R′ being as defined in claim 1.

9. The compound according to claim 8, wherein R′ is a substituted and unsubstituted aryl group.

10. The compound for the use according to claim 8, wherein R′ is a substituted or unsubstituted phenyl group.

11. The compound according to claim 8, wherein R₁ is chosen from groups having formula (B): 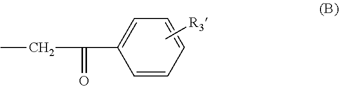
R′₃ being chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined in claim 1.

12. The compound according to claim 11, wherein R′₃ is a group in para position chosen from the alkyl groups comprising from 1 to 6 carbon atoms, and the groups having formula —OR′₄, R′₄ being chosen from H and alkyl groups comprising from 1 to 6 carbon atoms.

13. The compound according to claim 1, wherein R′ is a substituted and unsubstituted heteroaryl group.

14. The compound according to claim 13, wherein R₁ is chosen from the groups having the following formula (C): 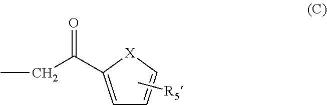
R′₅ being chosen from the group consisting of H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined in claim 1, and X being chosen from the group consisting of: S, O, and NH.

15. (canceled)

16. The compounds of claim 11, wherein R′₃ is in the para position.

17. The compound of claim 14, wherein X is S.

18. A method of treating bacterial infections in a patient in need thereof,
comprising
administering to said patient a therapeutically effective amount of a compound having formula (I): 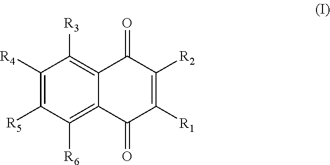
wherein:
R₁ is chosen from the group consisting of:
phenyl group, possibly substituted,
—CH₂—C₆H₄—R′₁ group, R′₁ being chosen from the group consisting of: H, —OH, halogen, alkyl, aryl, CHO, —CN, —NO₂, —SR_{α}, —OR_{α}, —NR_{α}R_{β}, —CONR_{α}R_{β}, —COOR_{α}, and —NHCOR_{α}, R_{α} and R_{β} representing independently from each other H, an alkyl group or an aryl group, R′₁ being preferably in para position, and
—CH₂—CO—R′ group, R′ representing an aryl or heteroaryl group, said aryl and heteroaryl groups being possibly substituted,
R₂ is chosen from the group consisting of: —OH and halogen,
R₃, R₄, R₅ and R₆ are chosen, independently from each other, in the group consisting of: H, —OH, halogen, alkyl, —CHO, —CN, —NO₂, —SR_{α}, —OR_{α}, —NR_{α}R_{β}, —COOR_{α}R_{β}, and —NHCOR_{α}, R_{α} and R_{β} being as defined above,
or its pharmaceutically acceptable salts, hydrates or hydrated salts or its polymorphic crystalline structures, racemates, diastereomers or enantiomers.

19. The method of claim 18, wherein said compound has formula (II): 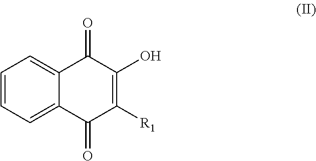
wherein R₁ is as defined in claim 18.

20. The method of claim 18, wherein R₁ is a substituted or unsubstituted phenyl group.

21. The method of claim 18, wherein R₁ is a phenyl group, substituted by an aryl group.

22. The method of claim 18, wherein R₁ is chosen from the group consisting of: —CH₂—C₆H₄—R′₁ group and —CH₂—CO—R′ group, R′₁ and R′ being as defined in claim 18.

23. The method of claim 18, wherein R₁ is chosen from the groups having the following formula (A): 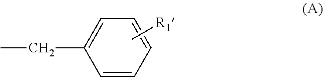
R′₁ being as defined in claim 18.

24. The method of claim 23, wherein R′₁ is a group in para position having formula —OR′₂, R′₂ being H or an alkyl group comprising from 1 to 6 carbon atoms.

25. The method of claim 18, wherein R₁ is chosen from the groups having formula —CH₂—CO—R′, R′ being as defined in claim 1.

26. The method of claim 25, wherein R′ is a substituted and unsubstituted aryl group.

27. The method of claim 25, wherein R′ is a substituted or unsubstituted phenyl group.

28. The method of claim 25, wherein R₁ is chosen from groups having formula (B): 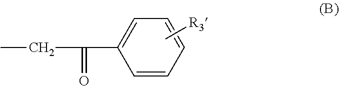
R′₃ being chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined in claim 1.

29. The method of claim 28, wherein R′₃ is a group in para position chosen from alkyl groups comprising from 1 to 6 carbon atoms, and groups having formula —OR′₄, R′₄ being chosen from H and alkyl groups comprising from 1 to 6 carbon atoms.

30. The method of claim 18, wherein R′ is chosen from substituted and unsubstituted heteroaryl groups.

31. The method of claim 30, wherein R₁ is chosen from the groups having the following formula (C): 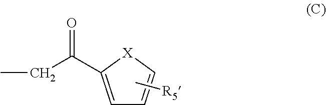
R′₅ being chosen from the group consisting of: H, —OH, halogen, alkyl, aryl, —CHO, —CN, —NO₂, —SR_{α}, —OR_{α}, —NR_{α}R_{β}, —CONR_{α}R_{β}, —COOR_{α}, and —NHCOR_{α}, R_{α} and R_{β} being as defined in claim 1, and X being chosen from the group consisting of: S, O, and NH.

32. The method of claim 28, wherein R′₃ is in the para position.

33. The method of claim 31, wherein X is S.

34. The method of claim 18, wherein the bacterial infections are Mycobacterium or Helicobacter species bacteria infections.

35. The method of claim 34, wherein the Mycobacterium infection is a Mycobacterium smegmatis infection.

36. The method of claim 34, wherein the Helicobacter infection is a Helicobacter pylori infection.
